(19) United States
(12) Patent Application Publication
(10) Pub. No.: US 2017/0273774 A1
(43) Pub. Date: Sep. 28, 2017

(54) Autonomous Stimulus Control Prosthetic

(71) Applicant: Kevin Mark Diaz, Dorchester, MA (US)

(72) Inventor: Kevin Mark Diaz, Dorchester, MA (US)

(21) Appl. No.: 15/083,004

(22) Filed: Mar. 28, 2016

(51) Int. Cl. A61F 2/02; A61N 1/05; A61N 1/36

(52) U.S. Cl. CPC A61F 2/022; A61N 1/36; A61N 1/05

(57) ABSTRACT

This operative technique allows for an alternative treatment for patients with diagnoses of hernias or other symptoms in various regions within the body. The proposed approach has the benefit of providing the surgeon faced with the lack of long-term treatment solutions for conditions and symptoms including neuromuscular disorders. The proposed prosthetic device and method presents surgeons with a multi-functional device for stimulus control, operative procedure and surgical technique to achieve results to relieve a variety of patient symptoms. This system provides an accurate robotic surgical technique allowing a minimally invasive procedure.

### BACKGROUND-PRIOR ART

The following is a tabulation of some prior art that presently appears relevant:

### U. S. Patent Applications

| Ser. No. | Publication Date | Applicant |
|---|---|---|
| US20040122526 | Jun. 24, 2004 | Imran Mir A. |
| US20040138760 | Jul. 15, 2004 | Schurr Marc O. |
| US20050283107 | Dec. 22, 2005 | Kalanovic Daniel |
| US20090138094 | May 28, 2009 | Schurr Marc O. |
| US20090299486 | Dec. 3, 2009 | Slimedics Ltd. |
| US20120095499 | Apr. 19, 2012 | Allergan, Inc. |
| US20120209400 | Aug. 16, 2012 | Schurr Marc O. |

### BACKGROUND

The operative technique presented allows for an alternative treatment for patients with diagnoses of hernias or similar symptoms in the abdominal region. The proposed approach has the benefit of providing the surgeon faced with the lack of long-term treatment solutions. Within this technology, the recent emerging scientific advances in the studies of neurophysiology and nanoscience are used and also may include microfluidic systems, biosensors and biochips as a platform for producing cellular and molecular biological solutions. In all populations, a percentage of adults develop and maintain conditions associated with the peripheral nervous system or hernia symptoms. These conditions are either chronic pain or mild visible physical hernia signs to meet a threshold of criteria for diagnosis to demand an innovative treatment.

These conditions are persistent for those patients where the signs of sleeplessness, pain lack of mobility, loss in ability in daily activities, to move or any other daily routine. This condition may also show symptoms at a variable state or as a chronic condition. This present technique allows for the General Physician to look for a category of symptoms to match a new diagnosis for successful treatments for patients having marked conditions of levels of pain where symptoms have been isolated to sensing organ dysfunction, increased intra-abdominal pressure or other organ and sensory discomfort. Closely related conditions match various diagnoses including the Spigelian condition in the space directly posterior to the external oblique muscle.

The present day capability to diagnose and provide treatment for this condition is very limited with most all diagnosis resulting in treatments for either constipation, enlarged prostate or other gastric processes not related to the underlying issues causing patient to seek successful minimally invasive long-term treatment. One new diagnosis and treatment is introduced as “Abdominal Fascia Resection” as an operative technique with an elastic and flexible space shaped prosthetic in order to provide General Physicians and Medical Practitioners the method for rapid identification of patients needing this functional long-term solution as a treatment.

The procedure may be performed either by open or laparoscopic methods with the closed method preferred for most patients in non-obese physical condition. Each patient can select which is their preference with the surgeon guiding the patient with recommendations of success and post-operative pain to be encountered and risks associated with each of the methods give the patient's conditions including abdominal wall structure and the severity of the existing conditions to be corrected. The prosthetic, FIG. (1), may also be utilized for various functions and include auxiliary systems used for needed daily adjustments for treatment of symptoms.

### SUMMARY

During the diagnosis stage, the patient's General Physician or other Medical Practitioner shall target the site where the symptoms are present in the patient along with the conditions which are reported. During this discovery phase suitable tests must be conducted in order to verify symptoms with physicals conditions which may be explained through physiology and histology known to science and medicine. These patient ailments may be directly related to either or both of the physical elements within the abdomen or the secondary Peripheral Nervous System (PNS). The results of tests such as CAT scan or other suitable test results localize and confirm the area in question as well as detailing where in the abdominal cavity the prevailing conditions exist. As these locations are discovered having divergence from proper anatomical form, structure and function, the relevant symptomatic organs in the viscera may be identified.

As an embodiment to the prosthetic's treatment delivery, the parasympathetic innervation process may be assisted in certain daily functions. Within the autonomic nervous system, the sympathetic, parasympathetic nervous system, the autonomic plexus and pelvic nerves are in control of daily functions of organs in the abdominal region. For each of the organs control, neurophysiology for the patient may be determined for any symptoms the patient may have with their particular organs. Having determined patient's problems with a diagnosis, the physician has the ability to conduct electrical impulses to affect locations to establish excitation with suitable nervous impulse signals. The reverse may be accomplished as well by reducing the action potential at areas where excitable membranes need to have impulse transmission absorbed into a membrane and be isolated locally to control the generation of undesirable stimuli.

The determination of the affected areas or herniated regions has a relationship with conditions for which the patient reports to the doctor. Those conditions are sensed as electric impulses from the PNS. In order to establish a surgical solution, the medical team must establish a treatment of the area in respect to all adjacent tissue physiology including the relationship for the internal position. These positions and orientations are viewed from each tissue's histology with respect to each structure characteristic and the threshold to carry mV potential. This nature of different tissue sensory capacity to carry various levels of electric nervous system impulse current is either at rest or antagonistic in relationship to the surrounding organs. It is this PNS relationship of tissue histology causing patient symptoms. The hypogastric and lumbar regions are defined boundaries where hernias occur. These locations where hernias form are further bounded by the transversalis fascia and to a lesser degree the aponeurosis. At these locations a primary factor causing stimulus is due to the excitations transmitting signals from gastro-intestinal processes which in-turn cause undesirable sensations and the patient's symptoms. These electric impulses are not normally present or sensed external to the abdomen. The nervous impulse transfers through to external skin possessing low membrane impedance and resistance.

Here, the operative technique provides a solution based upon these tissue characteristics and utilizes a properly devised biologic prosthetic inserted and attached to provide the separation of tissue in the visceroperitonel region or other to establish relief of patient symptoms and an effective treatment. This includes evaluating adjacent membrane surfaces at the affected location. Each membrane has an inherent ability for polarization where a membrane potential exists at the molecular level to form a mechanism of action to conduct and radiate electrical impulses towards the surface skin. This electric transfer mechanism is normally insulated by and within the surrounding tissue in the local surrounding viscera namely the peritoneum. Histology of the peritoneum is low with this additional layer thickness providing an insulation effect. Each affected membrane area has known voltage- or chemical-gated channels where ions conduct a potential difference. Different conductivity characteristics exist for each membrane and tissue. Engineering biocompatible composite membranes with polarization for grounding functions can facilitate or inhibit ion transmission and electrical conductance may be controlled.

As the entire transversalis fascia is evaluated for tissue physiology, the surgeon has the step of investigating for the determination for the correct placement of the bioprosthetic from the peritoneum in order to establish any of corrected position of viscera through resection. The normal section in this abdominal region has the boundary parietal peritoneum. Reestablishing the peritoneum boundary with adjacent viscera includes development from the point of view of normal cross-section anatomy. As the bioprosthetic has been sized to form the correct anatomical shape for the affected area, the surgeon must also evaluate the post-operative effects for physical activity. This includes the torso motion which has the extension and compressing effects within the abdominal area. The procedure for inserting and attaching the bioprosthetic in the defined area, compartment or space may include cautery and the use absorbable sutures which can minimize postoperative pain with low rate of recurrence likely for further procedures.

### DRAWINGS—FIGURES

FIG. 1 is a perspective view of the Prosthetic with extending flexible connectors.

### DRAWINGS REFERENCE NUMERALS

1 prosthetic surface 3 self-healing injection orifice

2 connectors

### DETAILED DESCRIPTION OF EMBODIMENTS

### Multi-Cavity Formable Bioprosthetic

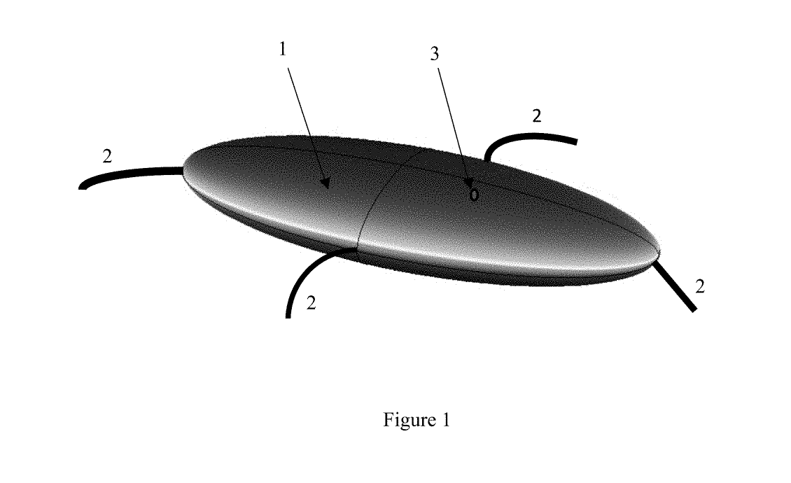

This bioproshetic may be standard size, configured with nanocomposites and have customized seamless design for specific patient needs. Each prosthetic has a number of variable or fixed length flexible and elastic connectors (2). Each connector end may have variable cross-section along its' length such as oval for band-like extensions. The connector and ends which connect to the chosen tissue may use conventional sutures, stem cells, semi-absorbable grafts, resorbable materials or utilize bio-nanotechnology connections comprised of any of the latest methods to connect tissues with prosthetics. The typical configuration is represented by the Perspective shown in FIG. 1, showing a Sample Prosthetic Shape with Connectors. The application of the bioprosthetic allows for a thickness to be established in the region were desired and may contain preformed shapes. These spaces are both shape and volume changing. The principle of utilizing prosthetics having a flexible and elastic external shell is two-fold. First, is the need for the prosthetic volume to compress. Secondly, the permanent prosthetic positioning demands prosthetic shell to contain flexible twisting characteristics similar to the actions of the human or animal torso. Each prosthetic may be rolled, twisted or folded and put into a sheath.

Each prosthetic surface (1) may be impermeable or permeable thickness, woven and have layers comprised of synthetic polymers, PDMS, platinum, natural biomaterials or biocompatible materials. Each surface may contain an internal insulating layer to establish a membrane potential. The prosthetic device may have internal diaphragms forming a space and cavities which may be filled with collagen or other appropriate material or fluid for the beneficial purpose of conducting electrical impulses and controlled with a micro-circuit and processor. Each of these prosthetic surfaces may also be used as a smaller membrane size as needed with or without internal cavities for impulse control including neuromuscular disorders. This method to autonomously regulate stimulus and secretions optimizes treatment efficacy.

For those prosthetic surfaces which require capability to control electrical impulses to and from the adjacent plasma-membrane, the membrane potential may be activated by electrical excitation by containing an intracellular conductive ion fluid control system. Layers may be biocompatible materials, utilize nanotechnology, polymers or nanocrystals having switch controlled surface conductivity characteristics across and along each side of the prosthetic layer. Multiple membrane layers may be used to achieve the desired ion flow, collection and to conduct electrical charge in the milli-volt range across membrane surfaces. Membrane edges may be connected in any way known to the art. These membranes may also form prosthetic connector ends of various sizes required to establish a pseudo-synaptic cleft and thus the needed nervous system control to transmit desired stimulus to achieve treatment of symptoms. Each of the membrane's resting potential is designed to be balanced. Histology has been established for the individual membranes and cells being connected to. Conductance may be reversible through the known extracellular spaces, openings, electrochemical channels, gates or the membrane. These surfaces may be used as smaller membranes, suitable attached at the distal ends of connectors and further connected to the affected organ requiring stimulus control through robotic surgical techniques. The prosthetic may be fabricated to fit contours to shape desired surfaces of internal spaces which are unique to each surgical procedure. An additional prosthetic mesh layer(s) or other prosthetic cross-section may extend from the edge of the prosthetic edge, be attached or be as one in an integrally fabricated structure using suitable manufacturing methods and techniques.

This prosthetic structure may additionally act as a module for housing of collecting electrical impulse energies with the use of capacitors and have circuits containing transducers, microchips and relays for release of said energy according to body orientation. Prosthetic structure walls may be comprised of conductive materials as a matrix to collect adjacent sensory electrical impulses and internally store energy potential into capacitors. To achieve sensory control, electrical impulses may be collected and controlled for any viscera timing objectives.

Timing control for the nighttime function of sleeping and daytime functions of both emptying bladder and moving bowels may be modulated. A ground may be utilized internally and the electrical energy stored in capacitors that may then be released in a controlled method through connectors and possibly into heat internally via a heat transfer method device to then stimulate organ functions. Contact points may be used as a network with polarity control by any manner known in the art including extending and attaching said connector ends to the natural sensory positions of desired organs. Wake and rest periods are optimized with stimulus control.

Highly precise bio-nanotechnology prosthetics with suture methods may be robotically implanted and attached allowing for connecting various devices. This is highly advantageous for each connector the prosthetic has to attach to an organ or to establish a fixed position within the abdominal or other region. Each connector may have a cross-section containing at least a central core for transmission with at least one internal conductive sensory impulse element. From the internal micro-circuits, connectors may extend to transmit electrical impulse delivery or collection to points or through graded potential membrane surfaces using an interconnected layered conductor series. This action potential across membranes allows for polarization control with voltage energy to affect ion flow transfer. Internal conductive elements may be a fluid to transmit charges through connectors and across membranes. Energy may be recharged and stored in an internal battery or be released externally with a controlled pulse with known resistance and timing sequence such as a Transcutaneous Energy Transfer (TET) device. Use of a grounding pad may also be used. Stored electrical energy may then be control released with microcircuits programmed for daily cycles of electrochemical collection and or release. Control may be established as well with horizontal and vertical orientation detection such as a micro-gyro or other suitable device. These electrical energy releases, diffusion and electrical gradient functions may be programmed externally by any wireless remote control method available in the art to modify and control function timing and release characteristics. This programming allows for the prosthetic device to automatically assist in daily organ and or muscular functions.

As another embodiment this prosthetic device may additionally house and control chemical absorption therapies to regulate the release for hormone therapy as a dosing mechanism or any other deficiency or surplus level where dosing with biochemical reaction agents are needed. This includes the use of permeable wall materials. Internal cavities may form a compartment for different functions including but not limited to dosing regulation and sensory assisting modules with connector networks. Each cavity may have a time release function internal to the prosthetic to provide needed ion transfer capability throughout the desired prosthetic surface utilizing microchannels, layers or other available methods known in the art. In this way the prosthetic allows for several applications to function in one prosthetic. Modern nanotechnology fabrication methods may be used to make customized prosthetic shapes including internal circuitry, microchips, solenoids, pumps, magnets, the attachment of connectors and ends with internal conductive elements. This shall provide one or multiple solutions for homeostasis of chronic problems involving any of the organ functions in the abdominal area or other area for improved daily living such as for metabolism regulation and hormone secretions.

In another embodiment, each cavity in the prosthetic may contain a specific volume of hormone or other fluid. The prosthetic may be positioned adjacent to the organ in need of supplemental control or stimulus added. The cavity volume may be activated for release by a micro-pump through the use of a computer. Internal diaphragms may compensate for volume contraction and expansion within the prosthetic cavities. For circumstances where the connector must deliver the stimulus or secretion, the connector element may have a central porous core to deliver required dosages along the connect length or to an end. Any method known in the art may be used as a gate or valve or switch to control either fluid or stimulus flow. One such method proposed is for use of proportionately sized in the prosthetic exterior wall or internal cavity wall having an internal passage size with passage gate which may be sensor activated which is sized to allow for precise flow based on the fluid's viscosity.

In another embodiment, prosthetics may be structures shaped and stiffened with biocompatible materials to form the needed dimensions for internal placement and to be externally attached and interconnected for articulating and extending modular robotic functions such as limbs, hands or devices for sensory collection and delivery processes and movements.

This operative technique proposes combining new and conventional Laparoscopic entry methods, insufflation and new biologic prosthetic design possibly comprising a module with microchips in order to achieve successful treatment. It is important to distinguish the point of the bioprosthetic to be inserted is not a conventional mesh as used and available in current hernia surgery procedures. These space prosthetic devices may be inserted via trocar anywhere for the benefit to implant internal electronic devices which may control organ and muscular actions. The details of Laparoscopic Operative Procedures vary for the position and size of prosthetic needed.

As a primary feature of design to the biologic prosthetic, the in-situ insertion includes the capability to expand in the peritoneum during insufflation through a removal from sheath process. As the surgeon has control of the prosthetic from both ends of the sheath, the position is held in place with the forceps and removed. Open repair surgery methods may be used as well. The connectors may be predesigned and arranged for use with resorbable or semi-absorbable ends and are then attached as planned for the final location of the biologic prosthetic. Cavities may be refilled by an injection orifice (3) on the prosthetic surface or into a connector end.

Post-operative treatment is required to establish both physiological normalization and psychological conditioning from the surgery's secondary effects such as closing issues of entry sites, surgical site infections (SSI), re-establishing activity, sleep restriction and a nutrition plan in order to develop new stability of the lower abdominal gastro-intestinal function. Patients must maintain daily and nightly eating and sleeping schedules to retrain the local internal gastro-intestinal organs which are adjusting to new orientations. The post-operative affect shall vary depending on the prosthetic options used and the severity of the existing conditions and for an adjustment period of adjacent viscera functioning in displaced positions. The most common adverse events for all surgical repair of hernias are pain, infection, hernia recurrence, scar-like tissue that sticks tissues together (adhesion), blockage of the large or small intestine (obstruction), bleeding, abnormal connection between organs, vessels, or intestines (fistula), fluid build-up, seroma at the surgical site and holes in adjacent tissues or organs (perforation).

The proposed prosthetic device and system provide surgeons a multi-functional device and operative procedure to achieve results and relieve a variety of patient symptoms. Multiple sensor elements within each connector and between more than one prosthetic device may be utilized for interconnection of devices to establish an internal feedback system to calibrate timing and allow for mnemonic data to be computed used for improved autonomous synergistic effects.

All embodiments, features and methods may be combined in any way to establish the desired device. This application description describes an apparatus, device and methods to control stimulus and perform surgical methods. Specific embodiments of this invention allow for numerous combinations for numerous additional advantages. Modifications and changes will readily occur to those skilled in the art without departing from the spirit and scope of this invention. This invention and its broader aspects are not limited to a specific detail and various modifications may be made without departing from the spirit or scope of the general inventive concept as defined by the appended claims and their equivalents.

I claim the following:

1) A prosthetic comprising at least two biocompatible surfaces and a space between said surfaces.

2) The apparatus of claim 1, further containing at least one connector extending from the side of said prosthetic.

3) The apparatus of claim 1, further comprising at least one connector having internal conductive elements to transfer electrical impulses.

4) The apparatus of claim 1, further comprising an energy source and micro-computer thereby autonomously regulating electrical stimulus.

5) The apparatus of claim 1, further comprising connecting an internal prosthetic cavity to at least one connector element containing an internal porous core for hormone delivery.

6) A surgical method combining inserting a prosthetic with defined space and attaching said prosthetic robotically.

7) The method of claim 6, further comprising attaching connectors to surfaces robotically, where said connectors transfer energy impulses through an internal conductive element.

8) The method of claim 6, further comprising attaching ends of connectors to surfaces robotically with suture material, where said connectors transfer energy impulses through an internal conductive element.
